(19) United States
(12) Patent Application Publication
(10) Pub. No.: US 2013/0309317 A1
(43) Pub. Date: Nov. 21, 2013

(54) FOOD SUPPLEMENT INCREASE CD34 STEM CELLS AND TELOMERASE ACTIVITY

(71) Applicants: HORNG-JYH HARN, HSINCHU COUNTY (TW); SHINN-ZONG LIN, HSINCHU COUNTY (TW); PO-CHENG LIN, HSINCHU COUNTY (TW); HSIN-I WANG, HSINCHU COUNTY (TW); PI-CHUN HUANG, HSINCHU COUNTY (TW)

(72) Inventors: HORNG-JYH HARN, HSINCHU COUNTY (TW); SHINN-ZONG LIN, HSINCHU COUNTY (TW); PO-CHENG LIN, HSINCHU COUNTY (TW); HSIN-I WANG, HSINCHU COUNTY (TW); PI-CHUN HUANG, HSINCHU COUNTY (TW)

(73) Assignee: GWO XI STEM CELL APPLIED TECHNOLOGY CO. LTD., HSINCHU COUNTY (TW)

(21) Appl. No.: 13/602,632

(22) Filed: Sep. 4, 2012

Related U.S. Application Data
(60) Provisional application No. 61/647,651, filed on May 16, 2012.

(30) Foreign Application Priority Data
Jul. 24, 2012 (TW) 101126668

(51) Int. Cl. A61K 35/50; A61P 35/00

(52) U.S. Cl. 424/583

(57) ABSTRACT

The present invention provides a food supplement, which comprises: a placental extract and an oil; which would promote the proliferation of CD34⁺ stem cells and increase the telomerase activity to achieve rejuvenation and anti-aging.

### CROSS-REFERENCE TO RELATED APPLICATIONS

This application claims the benefit of co-pending U.S. Provisional Application No. 61/647,651, filed May. 16, 2012, which is hereby incorporated herein.

### FIELD OF THE INVENTION

The present invention is related to a food supplement, which can induce proliferation of CD34⁺ stem cells and increase telomerase activity in the same time, so that the food supplement can contribute to rejuvenation and anti-aging.

### BACKGROUND OF THE INVENTION

In Korea and China, human placenta has been used as an anti-aging medicine for over many years, and it also can be used for wound healing. More recently, human placental extract was approved for life extension effect, and induces significant nitric oxide production, which is an important cellular mediator of tissue repair. Besides, it also contains many uncharacterized compounds believed to have various bioactivities involved in inhibition or delay of aging, inflammation, sunburn, mutagenicity, anaphylaxis, and oxidation it is been proved.

Hematopoietic stem cells (HSCs) represent a long-lived cell population that provides blood cells through hematopoiesis throughout the human lifespan. The cells are identified by the surface marker CD34⁺. Hematopoiesis is regulated by self-renewal, proliferation, and differentiation. In humans, CD34⁺ stem cells represent about 0.01% to 0.03% all cells in peripheral blood, which are very low concentration. Therefore, how to increase the CD34⁺ stem cells in the peripheral blood is the key factor of anti-aging. Furthermore, human cells progressively lose telomerase activity result in telomeric DNA is lost during successive rounds of DNA replication. Shortening of telomeres has been reported to correlate with cellular senescence.

Accordingly, the aim of the present invention is to provide food supplements which content placental extract to achieve the anti-aging effect by increasing the amount of CD34⁺ stem cells and the telomerase activity in peripheral blood.

### SUMMARY OF THE INVENTION

For the purpose, the present invention provides a food supplement, which can induce proliferation of CD34⁺ stem cells and increase telomerase activity in the same time, so that the food supplement can contribute to rejuvenation and anti-aging.

Therefore, the present invention provides a food supplement, which comprises: a placental extract; and an oil.

Preferably, the food supplement comprises 10%˜90% placental extract.

Preferably, the food supplement comprises 10%˜90% oil.

Preferably, the food supplement further comprises a royal jelly.

Preferably, the food supplement further comprises 10%˜80% royal jelly.

Preferably, the food supplement further comprises a carbohydrate.

Preferably, the food supplement further comprises 10%˜80% carbohydrate.

Preferably, the carbohydrate is dextran, L-fucose, ganoderma lucidum polysaccharide, mannose, fucose or trehalose.

Preferably, the placental extract is from a mammal.

Preferably, the mammal is a pig, a cattle, a sheep or a deer.

Preferably, the oil is selected from the group consisting of olive oil, evening primrose oil, soy bean oil, sesame oil, sunflower oil, brown-rice plumule oil, wheat plumule oil, avocado oil, safflower oil, tea seed oil, flax seed oil, perilla oil, corn oil, fish liver oil, fish oil and shrimp oil.

Preferably, the placental extract is placenta powder, placental extract solution, placental lyophilized powder or placental polypeptide.

Preferably, the food supplement is a soft capsule, a powder, a granule, a liquid, a gel or a paste.

Preferably, the food supplement can induce proliferation of CD34⁺ stem cells.

Preferably, the food supplement can increase telomerase activity in cells.

Preferably, the food supplement can be used in the proliferation of stem cells and anti-aging.

As mentioned above, the present invention provides food supplement which can induce proliferation of CD34⁺ stem cells and increase telomerase activity in the same time, so that the food supplement can contribute to rejuvenation and anti-aging.

### BRIEF DESCRIPTION OF THE DRAWINGS

FIG. 1 shows the change of CD34⁺ stem cell concentration in the peripheral blood of subjects who took or un-took the food supplement A of the present invention;

FIG. 2 shows the change of telomerase activity in the peripheral blood of subjects who took or un-took the food supplement A of the present invention.

### DETAILED DESCRIPTION OF THE INVENTION

The present invention provides a food supplement, which can induce proliferation of CD34⁺ stem cells and increase telomerase activity in the same time, so that the food supplement can contribute to rejuvenation and anti-aging.

The food supplement of present invention comprises a placental extract and an oil.

The ‘placental extract’ of the present invention can be all types of placental extract or placental product, which is comprising, but not limited to: placenta powder, placental extract solution, placental lyophilized powder or placental polypeptide. The placental extract is from a mammal, which is comprising, but not limited to: a pig, a cattle, a sheep or a deer. In the food supplement, the percentage of placental extract is 10%˜90%; in the best embodiment, the percentage of placental extract is 10%˜40%.

The ‘oil’ can be any suitable oil, including, but not limited to edible oil; In the best embodiment, it is selected from the group consisting of olive oil, evening primrose oil, soy bean oil, sesame oil, sunflower oil, brown-rice plumule oil, wheat plumule oil, avocado oil, safflower oil, tea seed oil, flax seed oil, perilla oil, corn oil, fish liver oil, fish oil and shrimp oil. In the food supplement, the percentage of the oil is 10%˜90%; in the best embodiment, which can include more than one kind of oil, and the percentage of the oil is 50%˜70%.

The food supplement of the present invention can further comprise a royal jelly. The ‘royal jelly’ can be all known royal jelly or other royal jelly product, which include, but not limited to royal jelly powder. In the food supplement, the percentage of the royal jelly or the royal jelly powder is 10%˜30%.

The food supplement of the present invention can further comprise a carbohydrate. The ‘carbohydrate’ includes dextran, L-fucose, ganoderma lucidum polysaccharide, mannose, fucose or trehalose. In the food supplement, the percentage of the carbohydrate is 10%˜30%.

The food supplement of the present invention can induce proliferation of CD34⁺ stem cells and also increase telomerase activity in the same time, so that the food supplement can contribute to rejuvenation and anti-aging.

### EXAMPLE 1

### Food Supplement A

The food supplement A of the present invention comprises:

(1) Pig placental powder 300 mg (30%);

(2) Avocado oil 300 mg (30%);

(3) Wheat plumule oil 300 mg (30%); and

(4) Royal jelly 100 mg (10%).

### EXAMPLE 2

### Food Supplement B

The food supplement B of the present invention comprises:

(1) Sheep placental dry powder 300 mg (30%);

(2) Royal jelly powder 100 mg (10%);

(3) Wheat plumule oil 300 mg (30%); and

(4) Avocado oil 300 mg (30%).

### EXAMPLE 3

### Food Supplement C

The food supplement C of the present invention comprises:

(1) Pig placental dry powder 200 mg (20%);

(2) Royal jelly powder 100 mg (10%);

(3) Wheat plumule oil 300 mg (30%);

(4) Avocado oil 300 mg (30%); and

(5) Dextran 100 mg (10%).

### EXAMPLE 4

### The Effect of Food Supplement a on Human Body

The food supplement A of the example 1 was tested to investigate the effect on CD34⁺ stem cells and telomerase activity in human.

The test was performed on 44 subjects, wherein 22 subjects are comparative group and the other 22 subjects are experimental group (taking the food supplement A of the present invention), after several months, the blood samples of the 44 subjects were investigated by following test and the results were showed as Table 1 (comparative group) and Table 2 (Experimental group).

TABLE 1
The test result of Comparative Group

| | | Gender | Intake | CD34⁺ | |
|---|---|---|---|---|---|
| Sample | Age | m = male | duration | cells | Telomerase |
| No. | (years) | f = female | (months) | (%) | activity |
| 1 | 19 | m | 0 | 0.10 | 0.14 |
| 2 | 20 | f | 0 | 0.10 | 0.08 |
| 3 | 20 | f | 0 | 0.20 | 0.08 |
| 4 | 21 | m | 0 | 0.20 | 0.10 |
| 5 | 21 | f | 0 | 0.20 | 0.08 |
| 6 | 22 | m | 0 | 0.30 | 0.08 |
| 7 | 32 | f | 0 | 0.70 | 0.15 |
| 8 | 39 | f | 0 | 0.50 | 0.09 |
| 9 | 41 | m | 0 | 0.10 | 0.13 |
| 10 | 46 | m | 0 | 0.20 | 0.13 |
| 11 | 47 | f | 0 | 0.30 | 0.15 |
| 12 | 48 | m | 0 | 0.10 | 0.10 |
| 13 | 53 | m | 0 | 0.20 | 0.08 |
| 14 | 53 | m | 0 | 0.30 | 0.08 |
| 15 | 54 | f | 0 | 0.20 | 0.11 |
| 16 | 56 | f | 0 | 0.30 | 0.10 |
| 17 | 56 | m | 0 | 0.05 | 0.11 |
| 18 | 57 | f | 0 | 0.10 | 0.09 |
| 19 | 58 | m | 0 | 0.50 | 0.12 |
| 20 | 60 | m | 0 | 0.50 | 0.08 |
| 21 | 64 | m | 0 | 0.15 | 0.09 |
| 22 | 64 | f | 0 | 0.05 | 0.08 |
| Aver- | 43 | 54.5% | | 0.24 | 0.10 |
| age | | male | | | |
| | | percentage | | | |
| SD | 15.8 | | | 0.17 | 0.02 |

TABLE 2
The test result of Experimental Group

| | | Gender | Intake | CD34⁺ | |
|---|---|---|---|---|---|
| Sample | Age | m = male | duration | cells | Telomerase |
| No. | (years) | f = female | (months) | (%) | activity |
| 23 | 27 | m | 2 | 4.60 | 0.27 |
| 24 | 27 | f | 2 | 3.90 | 0.12 |
| 25 | 35 | f | 2 | 1.35 | 0.14 |
| 26 | 39 | f | 8 | 1.70 | 0.14 |
| 27 | 40 | m | 12 | 1.70 | 0.13 |
| 28 | 41 | m | 3 | 1.00 | 0.10 |
| 29 | 43 | m | 12 | 0.60 | 0.08 |
| 30 | 44 | f | 5 | 0.70 | 0.09 |
| 31 | 44 | f | 6 | 0.30 | 0.09 |
| 32 | 45 | f | 1.7 | 5.95 | 0.10 |
| 33 | 46 | f | 2 | 0.20 | 0.12 |
| 34 | 46 | f | 8 | 0.40 | 0.23 |
| 35 | 46 | m | 23 | 1.70 | 0.22 |
| 36 | 48 | m | 1.2 | 0.30 | 0.13 |
| 37 | 49 | m | 14 | 0.35 | 0.15 |
| 38 | 51 | m | 12 | 1.25 | 0.13 |
| 39 | 54 | f | 12 | 0.30 | 0.12 |
| 40 | 57 | m | 12 | 1.10 | 0.10 |
| 41 | 58 | f | 8 | 1.10 | 0.08 |
| 42 | 58 | f | 11 | 0.60 | 0.11 |
| 43 | 59 | m | 2 | 0.65 | 0.10 |
| 44 | 61 | m | 3 | 1.50 | 0.15 |
| Aver- | 46 | 50% | | 1.42 | 0.13 |
| age | | male | | | |
| | | percentage | | | |
| SD | 9.2 | | | 1.47 | 0.05 |

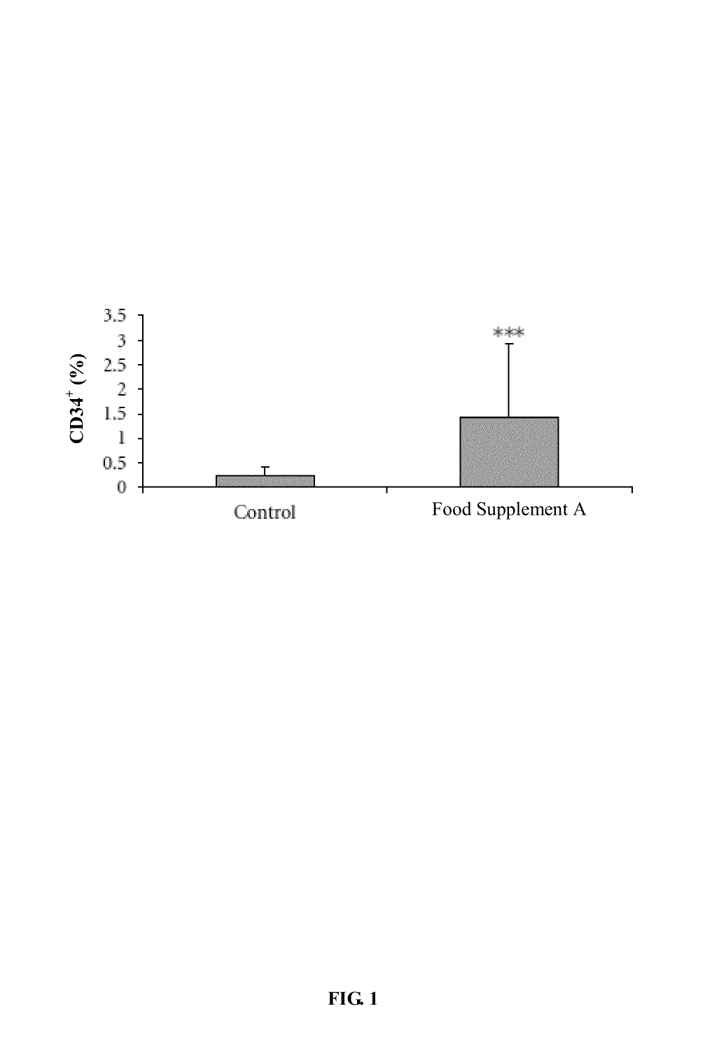

(1) The concentration of CD 34⁺ stem cells was investigated by flow cytometry. The result was showed as ‘CD 34⁺ cells (%)’ column in Table 1 and Table 2, which represented the percentage of CD 34⁺ cells in the peripheral blood. The result was also showed in FIG. 1, wherein the percentage of the CD 34⁺ stem cells in the peripheral blood from subjects who took the food supplement A was higher than those didn't take the food supplement A over 6 times. Moreover, the test was not affected by age, gender and intake duration.

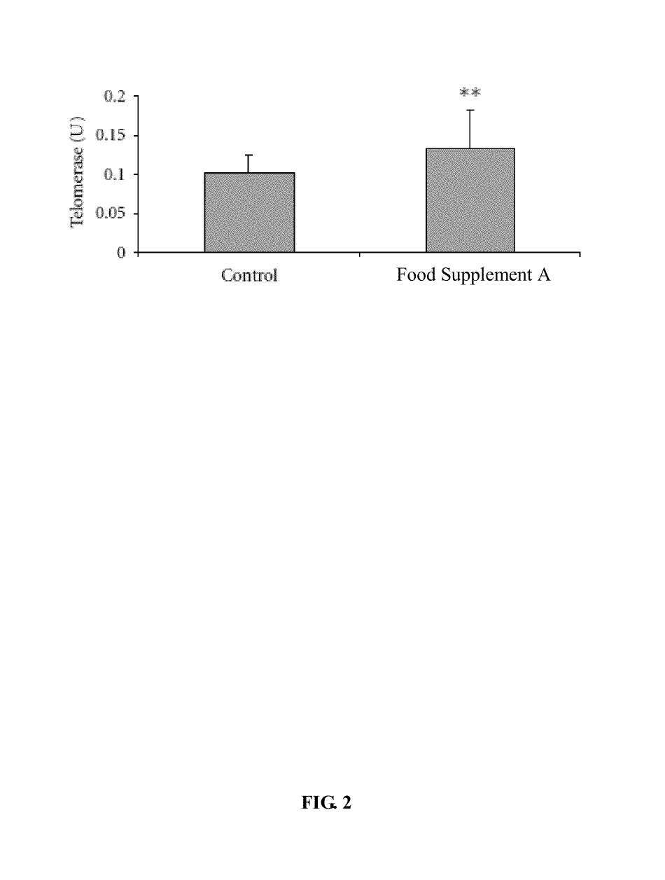

(2) The change of telomerase activity was investigated and the result was showed as ‘Telomerase activity’ column in Table 1 and Table 2, and also in FIG. 2. The telomerase activity in the peripheral blood from subjects who took the food supplement A was higher than those didn't take the food supplement A over 30%. Moreover, the test was not affected by age, gender and intake duration.

What is claimed is:

1. A food supplement, which comprises:
a placental extract; and
an oil.

2. The food supplement of claim 1, which comprises 10%˜90% placental extract.

3. The food supplement of claim 1, which comprises 10%˜90% oil.

4. The food supplement of claim 1, which further comprises a royal jelly.

5. The food supplement of claim 4, which further comprises 10%˜80% royal jelly.

6. The food supplement of claim 1, which further comprises a carbohydrate.

7. The food supplement of claim 6, which further comprises 10%˜80% carbohydrate.

8. The food supplement of claim 6, wherein the carbohydrate is dextran, L-fucose, ganoderma lucidum polysaccharide, mannose, fucose or trehalose.

9. The food supplement of claim 1, wherein the placental extract is from a mammal

10. The food supplement of claim 9, the mammal is a pig, a cattle, a sheep or a deer.

11. The food supplement of claim 1, wherein the oil is selected from the group consisting of olive oil, evening primrose oil, soy bean oil, sesame oil, sunflower oil, brown-rice plumule oil, wheat plumule oil, avocado oil, safflower oil, tea seed oil, flax seed oil, perilla oil, corn oil, fish liver oil, fish oil and shrimp oil.

12. The food supplement of claim 1, wherein the placental extract is placenta powder, placental extract solution, placental lyophilized powder or placental polypeptide.

13. The food supplement of claim 1, which is a soft capsule, a powder, a granule, a liquid, a gel or a paste.

14. The food supplement of claim 1, which can induce proliferation of CD34⁺ stem cells.

15. The food supplement of claim 1, which can increase telomerase activity in cells.

16. The food supplement of claim 1, which can be used in the proliferation of stem cells and anti-aging.
